(19) United States
(12) Patent Application Publication
(10) Pub. No.: US 2007/0060593 A1
(43) Pub. Date: Mar. 15, 2007

(54) 4-Carboxamido quinoline derivatives for use as nk-2 and nk-3

(76) Inventors: Jeffrey K. Kerns, KING OF PRUSSIA, PA (US); Hong Nie, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/561,745

(22) PCT Filed: Jun. 23, 2004
(86) PCT No.: PCT/US04/20333

§ 371 Date: Dec. 21, 2005

Related U.S. Application Data
(60) Provisional application No. 60/482,319, filed on Jun. 25, 2003.

(51) Int. Cl. A61K 31/496; C07D 403/02

(52) U.S. Cl. 514/253.06; 544/363

(57) ABSTRACT

Compounds of the formula (I) are disclosed which are NK2 and NK3 receptor antagonists and are useful in the treatment of respiratory diseases: 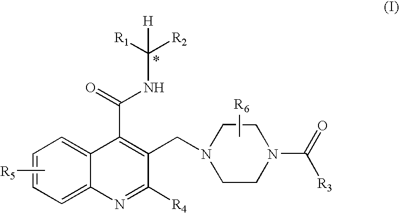 or a pharmaceutically acceptable salt thereof.

### FIELD OF THE INVENTION

The present invention relates to novel compounds, in particular to novel quinoline derivatives, to pharmaceutical compositions containing such compounds and to the use of such compounds in medicine.

### BACKGROUND OF THE INVENTION

The mammalian peptide Neurokinin B (NKB) belongs to the Tachykinin (TK) peptide family which also include Substance P (SP) and Neurokinin A (NKA). Pharmacological and molecular biological evidence has shown the existence of three subtypes of TK receptor (NK₁, NK₂ and NK₃) and NKB binds preferentially to the NK₃ receptor although it also recognizes the other two receptors with lower affinity (Maggi et al, 1993, J. Auton. Pharmacol., 13, 23-93).

Selective peptidic NK₃ receptor antagonists are known (Drapeau, 1990 Regul. Pept., 31, 125-135), and findings with peptidic NK₃ receptor agonists suggest that NKB, by activating the NK₃ receptor, has a key role in the modulation of neural input in airways, skin, spinal cord and nigro-striatal pathways (Myers and Undem, 1993, J. Physiol., 470, 665-679; Counture et al., 1993, Regul. Peptides, 46, 426-429; Mccarson and Krause, 1994, J. Neurosci., 14 (2), 712-720; Arenas et al. 1991, J. Neurosci., 11, 2332-8). However, the peptide-like nature of the known antagonists makes them likely to be too labile from a metabolic point of view to serve as practical therapeutic agents.

International Patent Application, Publication Number WO 00/58307 describes a series of aryl fused 2,4-disubstituted pyridines, such as naphthyridine derivatives, which are stated to exhibit biological activity as NK₃ receptor antagonists.

The compounds of the present invention are quinoline derivatives. Other quinoline derivatives have been described previously as selective NK₃ antagonists. For example, International Patent Application, Publication Numbers, WO 95/32948 and WO 96/02509 describe a series of selective and potent NK₃ receptor antagonists.

International Patent Application, Publication Number WO 00/64877 describes a series of 2-aminoquinolinecarboxamides as neurokinin receptor ligands.

International Patent Application, Publication Number, WO 00/58303 describes a series of 4-substituted quinoline derivatives which are stated to be NK₃ and/or GABA(A) receptor ligands. Such compounds are characterized by the presence of a nitrogen-containing heterocyclic moiety at the C(4) position of the quinoline ring.

International Patent Application, Publication Numbers, WO 97/21680, WO 98/52942, WO 00/31037, WO 00/31038, WO02/38547, WO 02/38548, WO 02/43734, WO 02/44154, and WO 02/44165 describe compounds which have biological activity as combined NK₃ and NK₂ receptor antagonists.

We have now discovered a further novel class of non-peptide NK₃ antagonists which are far more stable from a metabolic point of view than the known peptidic NK₃ receptor antagonists and are of potential therapeutic utility. These compounds also have NK₂ antagonist activity and are therefore considered to be of potential use in the prevention and treatment of a wide variety of clinical conditions, which are characterised by overstimulation of the Tachykinin receptors, in particular NK₃ and NK₂.

These conditions include respiratory diseases, such as chronic obstructive pulmonary disease (COPD), asthma, airway hyper-reactivity, cough; inflammatory diseases such as inflammatory bowel disease, psoriasis, fibrositis, osteoarthritis, rheumatoid arthritis and inflammatory pain; neurogenic inflammation or peripheral neuropathy, allergies such as eczema and rhinitis; ophthalmic diseases such as ocular inflammation, conjunctivitis, vernal conjuctivitis and the like; cutaneous diseases, skin disorders and itch, such as cutaneous wheal and flare, contact dermatitis, atopic dermatitis, urticaria and other eczematoid dermatitis; adverse immunological reactions such as rejection of transplanted tissues and disorders related to immune enhancement or suppression such as systhemic lupus erythematosis; gastrointestinal (GI) disorders and diseases of the GI tract such as disorders associated with the neuronal control of viscera such as ulcerative colitis, Crohn's disease, irritable bowel syndrome (IBS), gastro-exophageous reflex disease (GERD); urinary incontinence and disorders of the bladder function; renal disorders; increased blood pressure, proteinuria, coagulopathy and peripheral and cerebral oedema following pre-eclampsia in pregnancies (hereinafter referred to as the ‘Primary Conditions’).

Certain of these compounds also show CNS activity and hence are considered to be of particular use in the treatment of disorders of the central nervous system such as anxiety, depression, psychosis and schizophrenia; neurodegenerative disorders such as AIDS related dementia, senile dementia of the Alzheimer type, Alzheimer's disease, Down's syndrome, Huntingdon's disease, Parkinson's disease, movement disorders and convulsive disorders (for example epilepsy); demyelinating diseases such as multiple sclerosis and amyotrophic lateral sclerosis and other neuropathological disorders such as diabetic neuropathy, AIDS related neuropathy, chemotherapy-induced neuropathy and neuralgia; addiction disorders such as alcoholism; stress related somatic disorders; reflex sympathetic dystrophy such as shoulder/hand syndrome; dysthymic disorders; eating disorders (such as food intake disease); fibrosing and collagen diseases such as scleroderma and eosinophilic fascioliasis; disorders of the blood flow caused by vasodilatation and vasospastic diseases such as angina, migraine and Reynaud's disease and pain or nociception, for example, that is attributable to or associated with any of the foregoing conditions especially the transmission of pain in migraine, (hereinafter referred to as the ‘Secondary Conditions’).

The compounds of formula (I) are also considered to be useful as diagnostic tools for assessing the degree to which neurokinin-3 and neurokinin-2 receptor activity (normal, overactivity or underactivity) is implicated in a patients symptoms.

Certain compounds of the present invention have also been found to exhibit surprisingly advantageous pharmacochemical properties.

### DETAILED DESCRIPTION OF THE INVENTION

According to the present invention, there is provided a compound of formula (I) below or a pharmaceutically acceptable salt or solvate thereof: 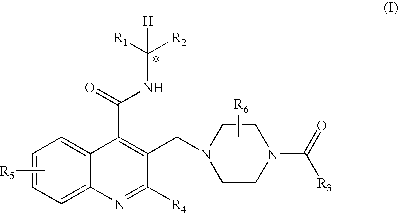
wherein:
R₁ is H or (C₁₋₆)alkyl;
R₂ is aryl, (C₃₋₇)cycloalkyl, or heterocycle; 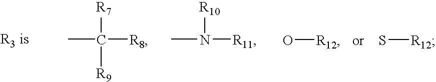
R₄ is phenyl or heterocycle;
R₅ is H or up to three substitutents independently selected from the list consisting of (C₁₋₆)alkyl, (C₂₋₆)alkenyl, aryl, alkoxy, or a hydroxylated deriviative thereof, hydroxy, halogen, nitro, cyano, carboxy, alkylcarboxy, alkylcarboxyalkyl, haloalkyl, and amino or mono- or dialkylamino; or R₅ represents a bridging moiety which is arranged to bridge, two adjacent ring atoms wherein the bridging moiety comprises alkyl or dioxyalkylene;
R₆ is absent or oxo;
R₇ is —OH or (C₁₋₆)alkylOH;
R₈ and R₉ are each independently H, (C₁₋₆)alkyl, (C₃₋₇)cycloalkyl, aryl, or heterocycle;
R₁₀ and R₁₁ together with the N atom form a heterocycle ring which is substituted by, —OH, or —C₁₋₆)alkylOH;
R₁₂ is H, (C₁₋₆)alkyl, aryl, or heterocycle; or a pharmaceutically acceptable salt thereof.
Suitably R₁ is methyl.
Suitably R₂ is (C₃₋₇)cycloalkyl.
Suitably R₃ is 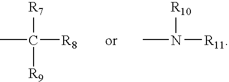
Suitably R₄ is heterocycle, more suitably 2- or 3-thiophene.
Suitably R₇ is —OH or (C₁₋₆)alkylOH unsubstituted or substituted by one to three halo groups.
Suitably R₈ and R₉ are each independently H, (C₁₋₆)alkyl, or (C₃₋₇)cycloalkyl. More suitably R₉ is H and R₈ is H, unsubstituted (C₃₋₇)cycloalkyl, or (C₁₋₆)alkyl unsubstituted or substituted by one to five substituents selected from the group consisting of halo and —H.
Suitably R₁₀ and R₁₁ together with the N atom form pyrrolidine substituted by —OH or -(C₁₋₆)alkylOH or piperidine substituted by —OH or —C₁₋₆)alkylOH.
Representative of the novel compounds of this invention are the following:
3-[4-(2-Hydroxy-ethanoyl)-piperazin-1-ylmethyl]-2-thiophen-2-yl-quinoline-4-carboxylic acid ((S)-1-cyclohexyl-ethyl)-amide;
3-[4-((S)-2-Hydroxy-propanoyl)-piperazin-1-ylmethyl]-2-thiophen-2-yl-quinoline-4-carboxylic acid ((S)-1-cyclohexyl-ethyl)-amide;
3-[4-(2-Hydroxy-2-methyl-propanoyl)-piperazin-1-ylmethyl]-2-thiophen-2-yl-quinoline-4-carboxylic acid ((S)-1-cyclohexyl-ethyl)-amide;
3-[4-((S)-2-Hydroxy-3-methyl-butanoyl)-piperazin-1-ylmethyl]-2-thiophen-2-yl-quinoline-4-carboxylicacid ((S)-1-cyclohexyl-ethyl)-amide;
3-[4-((S)-2-Cyclohexyl-2-hydroxy-ethanoyl)-piperazin-1-ylmethyl]-2-thiophen-2-yl-quinoline-4-carboxylic acid ((S)-1-cyclohexyl-ethyl)-amide;
3-{4-[1 -((R)-2-Hydroxymethyl-pyrrolidin-1-yl)-methanoyl]-piperazin-1-ylmethyl}-2-thiophen-2-yl-quinoline-4-carboxylic acid ((S)-1-cyclohexyl-ethyl)-amide;
3-{4-[1-((S)-2-Hydroxymethyl-pyrrolidin-1-yl)-methanoyl]-piperazin-1-ylmethyl}-2-thiophen-2-yl-quinoline-4-carboxylic acid ((S)-1-cyclohexyl-ethyl)-amide;
3-{4-[1 -(4-Hydroxy-piperidin-1-yl)-methanoyl]-piperazin-1-ylmethyl}-2-thiophen-2-yl-quinoline-4-carboxylic acid ((S)-1-cyclohexyl-ethyl)-amide;
2-Thiophen-2-yl-3-[4-(3,3,3-trifluoro-2-hydroxy-2-methyl-propanoyl)-piperazin-1-ylmethyl]-quinoline-4-carboxylic acid ((S)-1-cyclohexyl-ethyl)-amide;
2-Thiophen-2-yl-3-[4-(3,3,3-trifluoro-2-hydroxy-propanoyl)-piperazin-1-ylmethyl]-quinoline-4-carboxylic acid ((S)-1-cyclohexyl-ethyl)-amide;
2-Thiophen-2-yl-3-[4-(4,4,4-trifluoro-3-hydroxy-3-methyl-butahoyl)-piperazin-1-ylmethyl]-quinoline-4-carboxylic acid ((S)-1-cyclohexyl-ethyl)-amide;
2-Thiophen-2-yl-3-[4-(4,4,4-trifluoro-3-hydroxy-butanoyl)-piperazin-1-ylmethyl]-quinoline-4-carboxylic acid ((S)-1-cyclohexyl-ethyl)-amide; and
3-[4-((S)-2-Hydroxy-propanoyl)-2-oxo-piperazin-1-ylmethyl]-2-thiophen-2-yl-quinoline-4-carboxylic acid ((S)-1-cyclohexyl-ethyl)-amide; or a pharmaceutically acceptable salt thereof.

The compounds of formula (I) may have at least one asymmetric centre—for example the carbon atom labelled with an asterisk (*) in the compound of formula (I)—and therefore may exist in more than one stereoisomeric form. The invention extends to all such stereoisomeric forms and to mixtures thereof, including racemates. In particular, the invention includes compounds wherein the asterisked carbon atom in formula (I) has the stereochemistry shown in formula (Ib): 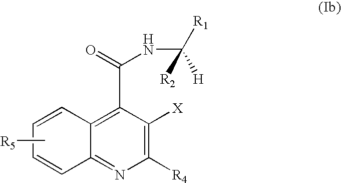
wherein R₁, R₂, R₄, and R₅ are as defined in relation to formula (I), and X represents the moiety 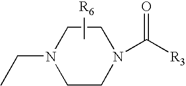
wherein R₆ and R₃ are as defined in relation to formula (I).

The compounds of formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels.

A substantially pure form will generally contain at least 50% (excluding normal pharmaceutical additives), preferably 75%, more preferably 90% and still more preferably 95% of the compound of formula (I) or its salt or solvate.

One preferred pharmaceutically acceptable form is the crystalline form, including such form in pharmaceutical composition. In the case of salts and solvates the additional ionic and solvent moieties must also be non-toxic.

Suitable salts are pharmaceutically acceptable salts.

Suitable pharmaceutically acceptable salts include the acid addition salts with the conventional pharmaceutical acids, for example maleic, hydrochloric, hydrobromic, phosphoric, acetic, trifluoroacetic, fumaric, salicylic, citric, lactic, mandelic, tartaric, succinic, benzoic, ascorbic and methanesulphonic.

Suitable pharmaceutically acceptable salts include salts of acidic moieties of the compounds of formula (I) when they are present, for example salts of carboxy groups or phenolic hydroxy groups.

Suitable salts of acidic moieties include metal salts, such as for example aluminium, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-β-phenethylamine, dehydroabietylamine, N,N′-bisdehydroabietylamine, glucamine, N-methylglucamine or bases of the pyridine type such as pyridine, collidine, quinine or quinoline.

Suitable solvates are pharmaceutically acceptable solvates.

Suitable pharmaceutically acceptable solvates include hydrates.

Unless otherwise defined, the term (C₁₋₆)alkyl when used alone or when forming part of other groups (such as the ‘(C₁₋₆)alkylOH’ group) includes substituted or unsubstituted, straight or branched chain alkyl groups containing 1 to 6 carbon atoms. Examples of (C₁₋₆)alkyl groups include methyl, ethyl, n-propyl, isopropyl, n-butyl, isobutyl, t-butyl, n-pentyl, isopentyl, neopentyl, and hexyl.

The term (C₂₋₆)alkenyl means a substituted or unsubstituted alkyl group of 2 to 6 carbon atoms, wherein one carbon-carbon single bond is replaced by a carbon-carbon double bond. Examples of (C₂₋₆)alkenyl include ethylene, 1-propene, 2-propene, 1-butene, 2-butene, and isobutene. Both cis and trans isomers are included.

The term (C₃₋₇)cycloalkyl refers to subsituted or unsubstituted carbocyclic ring system of three to seven carbon atoms, which may contain up to two unsaturated carbon-carbon bonds. Examples of (C₃₋₇)cycloalkyl include cyclopropyl, cyclobutyl, cyclopentyl, cyclopentenyl, cyclohexyl, cyclohexenyl, and cycloheptyl.

Unless otherwise defined, suitable substituents for any (C₁₋₆)alkyl, (C₂₋₆)alkenyl, and (C₃₋₇)cycloalkyl group, when used alone or when forming part of other groups (such as the ‘(C₁₋₆)alkylOH’ group), includes up to five substituents, which may be on any carbon atom that results in a stable structure and is available by conventional synthetic techniques. Suitable substituents are halo, —OR′, —SR′, (C₁₋₆)alkylsulfonyl, (C₁₋₆)alkylsulfoxyl, —N(R′)₂, —CH₂N(R′)₂, nitro, cyano, —CO₂R′, —CON(R′)₂, —COR′, and

—NR′ C(O)R′, wherein each R′ is independently H or unsubstituted (C₁₋₆)alkyl.

Halo or halogen includes fluoro, chloro, bromo and iodo.

Ar or aryl, as applied herein, means phenyl or naphthyl, or phenyl or naphthyl substituted by one to three substituents, which may be on any carbon atom that results in a stable structure and is available by conventional synthetic techniques. Suitable substituents are halo, —OR′, —SR′, (C₁₋₆)alkylsulfonyl, (C₁₋₆)alkylsulfoxyl, —N(R′)₂, —CH₂N(R′)₂, nitro, cyano, —CO₂R′, —CON(R′)₂, —COR′, and —NR′C(O)R′, wherein each R′ is independently H or unsubstituted (C₁₋₆)alkyl.

The term ‘het’ or ‘heterocycle’ indicates a unsubstituted or substituted five or six membered monocyclic ring, or a nine or ten membered bicyclic ring containing one to three heteroatoms chosen from the group of nitrogen, oxygen, and sulfur, which is stable and available by conventional chemical synthesis. Illustrative heterocycles are benzofuran, benzimidazole, benzopyran, benzothiophene, benzothiazole, furan, imidazole, indoline, morpholine, piperidine, piperazine, pyrrole, pyrrolidine, tetrahydropyridine, pyridine, thiazole, oxazole, thiophene, quinoline, isoquinoline, pyrrolidine, pyridine, and piperizine. Unless otherwise defined, any heterocycle group contains up to three substitutents selected from the group of halo, —OR′, —SR′, (C₁₋₆)alfkylsulfonyl, (C₁₋₆)alkylsulfoxyl, —N(R′)₂, —CH₂N(R′)₂, nitro, cyano, —CO₂R′, —CON(R′)₂, —COR′, and —NR′ C(O)R′, wherein each R′ is independently H or unsubstituted (C₁₋₆)alkyl.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical, Bn refers to the benzyl radical, Me refers to methyl, Et refers to ethyl, Ac refers to acetyl, Alk refers to C₁₋₄alkyl, Nph refers to 1- or 2-naphthyl and cHex refers to cyclohexyl. Tet refers to 5-tetrazolyl.

Certain reagents are abbreviated herein. DCC refers to dicyclohexylcarbodiimide, DMAP refers to dimethylaminopyridine, DIEA refers to diisopropylethyl amine, EDC refers to 1-(3-dimethylaminopropyl)-3-ethylcarbodiimide, hydrochloride. HOBt refers to 1-hydroxybenzotriazole, THF refers to tetrahydrofuran, DIEA refers to diisopropylethylamine, DEAD refers to diethyl azodicarboxylate, PPh₃ refers to triphenylphosphine, DIAD refers to diisopropyl azodicarboxylate, DME refers to dimethoxyethane, DMF refers to dimethylformamide, NBS refers to N-bromosuccinimide, Pd/C refers to a palladium on carbon catalyst, PPA refers to polyphosphoric acid, DPPA refers to diphenylphosphoryl azide, BOP refers to benzotriazol-1-yloxy-tris(dimethyl-amino)phosphonium hexafluorophosphate, HF refers to hydrofluoric acid, TEA refers to triethylamine, TFA refers to trifluoroacetic acid, PCC refers to pyridinium chlorochromate.

Compounds of the formula (I) are prepared by the general methods discribed in Schemes 1 and 2. 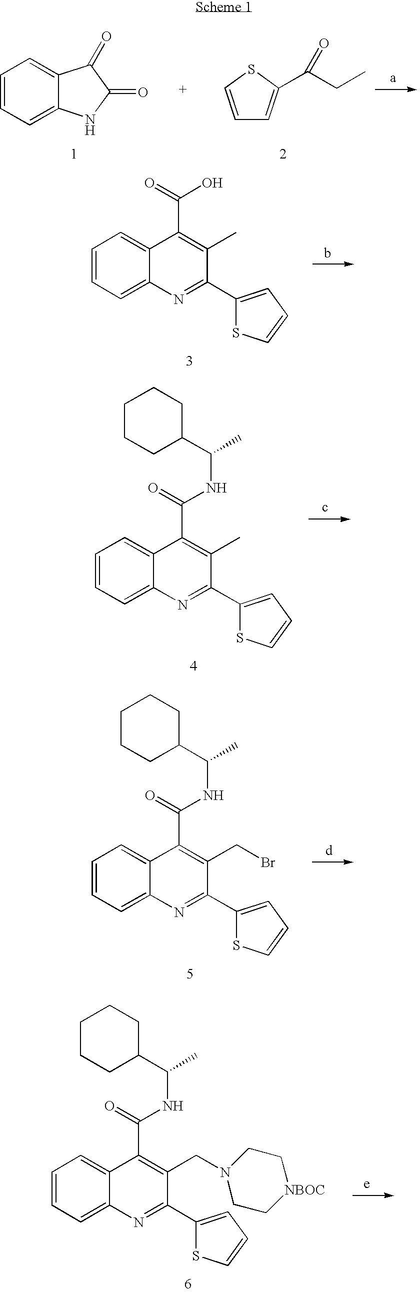 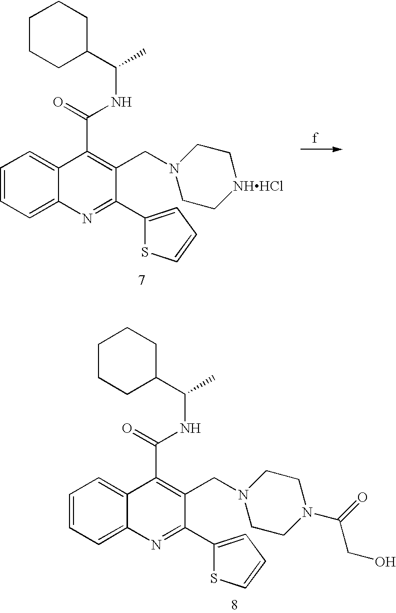
Reagents and Conditions: a) KOH, EtOH; b) Oxallyl chloride, DMF (cat.) CH₂Cl₂; (S)-Cyclohexylethylamine, triethylamine, CH₂Cl₂; c) NBS, dibenzoyl peroxide, CCl₄; d) tert-Butyl 1-piperazinecarboxylate, potassium carbonate, CH₃CN; e) HCl, dioxane; f) Hydroxy-acetic acid, EDC, HOBt, Et₃N, CH₂Cl₂.

Compounds of the general formula (I) may be prepared as is depicted in Scheme 1. Thus, reaction of 1-(2-thienyl)-1-propanone with isatin under basic conditions yields the desired carboxylic acid 3. Conversion to the acid chloride followed by reaction with S-(—)-1-cyclohexylethylamine produces amide 4. This in turn is converted to 3-piperazin-1-ylmethyl-2-thiophen-2-yl-quinoline-4-carboxylic acid ((S)-1-cyclohexyl-ethyl)-amide (6) (WO0244165) via the two step procedure of free radical bromination followed by S_{N}2 displacement with tert-butyl 1-piperazinecarboxylate. Removal of the BOC protecting group under acidic conditions followed by coupling with the appropriate carboxylic acid yields the desired amide 8. 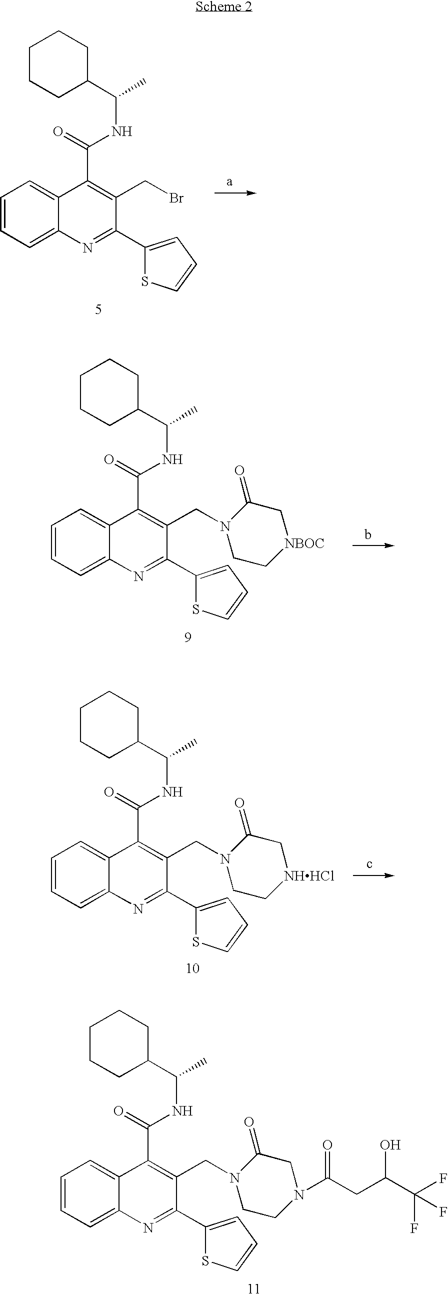
Reagents and Conditions: a) NaH, 3-oxo-piperazine-1-carboxylic acid tert-butylester, DMF/DMSO, 0° C.; b) HCl, dioxane; c) 4,4,4-trifluoro-3-hydroxy-butyric acid, EDC, HOBt, triethylamine, CH₂Cl₂.

Alternatively, compounds of formula (I) may be prepared in a fashion analogous to that depicted in Scheme 2. Thus, S_{N}2 displacement of the quinolinyl bromide 5 with 3-oxo-piperazine-1-carboxylic acid tert-butyl ester under basic conditions affords BOC carbamate 9. Removal of the BOC protecting group under acidic conditions followed by coupling of the product with the appropriate carboxylic acid yields the desired amide 11.

Accordingly the present invention also provides a compound of formula (I), or a pharmaceutically acceptable salt or solvate thereof, for use as an active therapeutic substance.

In particular, the present invention also provides a compound of formula (I), or a pharmaceutically acceptable salt or solvate thereof, for the treatment or prophylaxis of the Primary and Secondary Conditions.

The present invention further provides a pharmaceutical composition comprising a compound of formula (I), or a pharmaceutically acceptable salt or solvate thereof, and a pharmaceutically acceptable carrier.

The present invention also provides the use of a compound of formula (I), or a pharmaceutically acceptable salt or solvate thereof, in the manufacture of a medicament for the treatment of the Primary and Secondary Conditions.

As mentioned above the Primary conditions include respiratory diseases, such as chronic obstructive pulmonary disease (COPD), asthma, airway hyperreactivity, cough; inflammatory diseases such as inflammatory bowel disease, psoriasis, fibrositis, osteoarthritis, rheumatoid arthritis and inflammatory pain; neurogenic inflammation or peripheral neuropathy, allergies such as eczema and rhinitis; ophthalmic diseases such as ocular inflammation, conjunctivitis, vernal conjuctivitis and the like; cutaneous diseases, skin disorders and itch, such as cutaneous wheal and flare, contact dermatitis, atopic dermatitis, urticaria and other eczematoid dermatitis; adverse immunological reactions such as rejection of transplanted tissues and disorders related to immune enhancement or suppression such as systhemic lupus erythematosis; gastrointestinal (GI) disorders and diseases of the GI tract such as disorders associated with the neuronal control of viscera such as ulcerative colitis, Crohn's disease, irritable bowel syndrome (IBS), gastro-exophageous reflex disease (GERD); urinary incontinence and disorders of the bladder function; renal disorders.

As mentioned above, the Secondary conditions disorders of the central nervous system such as anxiety, depression, psychosis and schizophrenia; neurodegenerative disorders such as AIDS related dementia, senile dementia of the Alzheimer type, Alzheimer's disease, Down's syndrome, Huntington's disease, Parkinson's disease, movement disorders and convulsive disorders (for example epilepsy); demyelinating diseases such as multiple sclerosis and amyotrophic lateral sclerosis and other neuropathological disorders such as diabetic neuropathy, AIDS related neuropathy, chemotherapy-induced neuropathy and neuralgia; addiction disorders such as alcoholism; stress related somatic disorders; reflex sympathetic dystrophy such as shoulder/hand syndrome; dysthymic disorders; eating disorders (such as food intake disease); fibrosing and collagen diseases such as scleroderma and eosinophilic fascioliasis; disorders of the blood flow caused by vasodilation and vasospastic diseases such as angina, migraine and Reynaud's disease and pain or nociception, for example, that is attributable to or associated with any of the foregoing conditions especially the transmission of pain in migraine.

Such a medicament, and a composition of this invention, may be prepared by admixture of a compound of the invention with an appropriate carrier. It may contain a diluent, binder, filler, disintegrant, flavouring agent, colouring agent, lubricant or preservative in conventional manner.

These conventional excipients may be employed for example as in the preparation of compositions of known agents for treating the conditions.

Preferably, a pharmaceutical composition of the invention is in unit dosage form and in a form adapted for use in the medical or veterinarial fields. For example, such preparations may be in a pack form accompanied by written or printed instructions for use as an agent in the treatment of the conditions.

The suitable dosage range for the compounds of the invention depends on the compound to be employed and on the condition of the patient. It will also depend, inter alia, upon the relation of potency to absorbability and the frequency and route of administration.

The compound or composition of the invention may be formulated for administration by any route, and is preferably in unit dosage form or in a form that a human patient may administer to himself in a single dosage. Advantageously, the composition is suitable for oral, rectal, topical, parenterai, intravenous or intramuscular administration. Preparations may be designed to give slow release of the active ingredient.

Compositions may, for example, be in the form of tablets, capsules, sachets, vials, powders, granules, lozenges, reconstitutable powders, or liquid preparations, for example solutions or suspensions, or suppositories.

The compositions, for example those suitable for oral administration, may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinyl-pyrrolidone, sodium starch glycollate or microcrystalline cellulose; or pharmaceutically acceptable setting agents such as sodium lauryl sulphate.

Solid compositions may be obtained by conventional methods of blending, filling, tabletting or the like. Repeated blending operations may be used to distribute the active agent throughout those compositions employing large quantities of fillers. When the composition is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The composition may also be in the form of an ingestible capsule, for example of gelatin containing the compound, if desired with a carrier or other excipients.

Compositions for oral administration as liquids may be in the form of, for example, emulsions, syrups, or elixirs, or may be presented as a dry product for reconstitution with water or other suitable vehicle before use. Such liquid compositions may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; aqueous or non-aqueous vehicles, which include edible oils, for example almond oil, fractionated coconut oil, oily esters, for example esters of glycerine, or propylene glycol, or ethyl alcohol, glycerine, water or normal saline; preservatives, for example methyl or propyl p-hydroxybenzoate or sorbic acid; and if desired conventional flavouring or colouring agents.

The compounds of this invention may also be administered by a non-oral route. In accordance with routine pharmaceutical procedure, the compositions may be formulated, for example for rectal administration as a suppository. They may also be formulated for presentation in an injectable form in an aqueous or non-aqueous solution, suspension or emulsion in a pharmaceutically acceptable liquid, e.g. sterile pyrogen-free water or a parenterally acceptable oil or a mixture of liquids. The liquid may contain bacteriostatic agents, anti-oxidants or other preservatives, buffers or solutes to render the solution isotonic with the blood, thickening agents, suspending agents or other pharmaceutically acceptable additives. Such forms will be presented in unit dose form such as ampoules or disposable injection devices or in multi-dose forms such as a bottle from which the appropriate dose may be withdrawn or a solid form or concentrate which can be used to prepare an injectable formulation.

The compounds of this invention may also be administered by inhalation, via the nasal or oral routes. Such administration can be carried out with a spray formulation comprising a compound of the invention and a suitable carrier, optionally suspended in, for example, a hydrocarbon propellant.

Preferred spray formulations comprise micronised compound particles in combination with a surfactant, solvent or a dispersing agent to prevent the sedimentation of suspended particles. Preferably, the compound particle size is from about 2 to 10 microns.

A further mode of administration of the compounds of the invention comprises transdermal delivery utilising a skin-patch formulation. A preferred formulation comprises a compound of the invention dispersed in a pressure sensitive adhesive which adheres to the skin, thereby permitting the compound to diffuse from the adhesive through the skin for delivery to the patient. For a constant rate of percutaneous absorption, pressure sensitive adhesives known in the art such as natural rubber or silicone can be used.

As mentioned above, the effective dose of compound depends on the particular compound employed, the condition of the patient and on the frequency and route of administration. A unit dose will generally contain from 20 to 1000 mg and preferably will contain from 30 to 500 mg, in particular 50, 100, 150, 200, 250, 300, 350, 400, 450, or 500 mg. The composition may be administered once or more times a day for example 2, 3 or 4 times daily, and the total daily dose for a 70 kg adult will normally be in the range 100 to 3000 mg. Alternatively the unit dose will contain from 2 to 20 mg of active ingredient and be administered in multiples, if desired, to give the preceding daily dose.

No unacceptable toxicological effects are expected with compounds of the invention when administered in accordance with the invention.

The present invention also provides a method for the treatment and/or prophylaxis of the Primary and Secondary Conditions in mammals, particularly humans, which comprises administering to the mammal in need of such treatment and/or prophylaxis an effective, non-toxic pharmaceutically acceptable amount of a compound of formula (I) or a pharmaceutically acceptable salt or solvate thereof.

The activity of the compounds of the present invention, as NK₃ ligands, is determined by their ability to inhibit the binding of the radiolabelled NK₃ ligands, [¹²⁵I]-[Me-Phe⁷]-NKB or [³H]-Senktide, to guinea-pig and human NK₃ receptors (Renzetti et al, 1991, Neuropeptide, 18, 104-114; Buell et al, 1992, FEBS, 299(1), 90-95; Chung et al, 1994, Biochem. Biophys. Res. Commun., 198(3), 967-972).

The binding assays utilized allow the determination of the concentration of the individual compound required to reduce by 50% the [¹²⁵I]-[Me-Phe⁷]-NKB and [³H]-Senktide specific binding to NK₃ receptor in equilibrium conditions (IC50).

Binding assays provide for each compound tested a mean IC₅₀ value of 2-5 separate experiments performed in duplicate or triplicate. The most potent compounds of the present invention show IC₅₀ values in the range 10-1000 nM. The NK₃-antagonist activity of the compounds of the present invention is determined by their ability to inhibit senktide-induced contraction of the guinea-pig ileum (Maggi et al, 1990, Br. J. Pharmacol., 101, 996-1000) and rabbit isolated iris sphincter muscle (Hall et al., 1991, Eur. J. Pharmacol., 199, 9-14) and human NK₃ receptors-mediated Ca⁺⁺ mobilization (Mochizuki et al, 1994, J. Biol. Chem., 269, 9651-9658). Guinea-pig and rabbit in-vitro functional assays provide for each compound tested a mean K_{B} value of 3-8 separate experiments, where K_{B} is the concentration of the individual compound required to produce a 2-fold rightward shift in the concentration-response curve of senktide. Human receptor functional assay allows the determination of the concentration of the individual compound required to reduce by 50% (IC₅₀ values) the Ca⁺⁺ mobilization induced by the agonist NKB. In this assay, the compounds of the present invention behave as antagonists.

Alternatively, the binding assay may be performed as follows: ¹²⁵I-NKA and ¹²⁵I-[MePhe7]-NKB (PerkinElmer) were used in the binding Scintillation proximity assay (SPA) of NK2 and NK3 receptor, respectively. Polystrene Leadseeker WGA-SPA beads (Amersham Biosciences) was mixed with plasma membrane prepared from CHO cell lines expressing NK2 or NK3 in a bead/membrane ratio of 20:1 (w/w) in assay buffer (75 mM Tris pH 7.8, 75 mM NaCl, 4 mM MnCl2, 1 mM EDTA, 0.05% Chaps, 1 mM PMSF). The mixture was placed on ice for 30 minutes to allow the formation of membrane/bead complex before BSA was added to a final concentration of 1%. After another 30 minutes incubation on ice, the bead/membrane complex was washed twice and suspended in assay buffer. ¹²⁵I-labeled ligands were then added to the bead/membrane complex. 30 uL of the resulting mixture is then dispensed to each well of Nalgen NUNC 384-well plate with 1 uL compound pre-dispensed in DMSO. The plates were then sealed and pulse spin at 1100 rpm. After 3 hours incubation at room temperature with shaking, the plates were spin for 2 min at 1100 rpm and measured in Viewlux Plus imager (PerkinElmer) for 2×5 minutes with a 618-nm filter. Inhibition of radioactive ligand binding to it respective receptor was measured by the reduction of signal. IC50 of each compound was determined by an 11-point 3×-dilution inhibition curve. pKi was calculated using Kd of each radioactive ligand determined in a separate experiment.

The therapeutic potential of the compounds of the present invention in treating the conditions can be assessed using rodent disease models.

As stated above, the compounds of formula (I) are also considered to be useful as diagnostic tool. Accordingly, the invention includes a compound of formula (I) for use as diagnostic tools for assessing the degree to which neurokinin-2 and neurokinin-3 receptor activity (normal, overactivity or underactivity) is implicated in a patients symptoms. Such use comprises the use of a compound of formula (I) as an antagonist of said activity, for example including but not restricted to tachykinin agonist-induced inositol phosphate turnover or electrophysiological activation, of a cell sample obtained from a patient. Comparison of such activity in the presence or absence of a compound of formula (I), will disclose the degree of NK-2 and NK-3 receptor involvement in the mediation of agonist effects in that tissue.

### Descriptions and Experimental

Nuclear magnetic resonance spectra were recorded at 400 MHz using a Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (δ) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API III, or VG ZAB HF instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel.

### EXAMPLES

In the following synthetic examples, temperature is in degrees Centigrade (° C.). Unless otherwise indicated, all of the starting materials were obtained from commercial sources. For reverse phase HPLC (unless otherwise stated), a 50×20 mm I.D. YMC CombiPrep ODS-A column at 20 mL/min with a 10 min gradient from 10% CH₃CN to 90% CH₃CN in H₂O was used with a 2 min hold at 90% CH₃CN in H₂O at the end of each run. Without further elaboration, it is believed that one skilled in the art can, using the preceding description, utilize the present invention to its fullest extent. These Examples are given to illustrate the invention, not to limit its scope.

### Example 1

### Preparation of 3-[4-(2-hydroxy-ethanoyl)-piperazin-1-ylmethyl]-2-thiophen-2-yl-quinoline-4-carboxylic acid ((S)-1-cyclohexyl-ethyl)-amide

1a) 3-Bromomethyl-2-thiophen-2-yl-quinoline-4-carboxylic acid, (S)-(1-cyclohexylethyl)amide

Methyl-2-thiophen-2-yl-quinoline-4-carboxylic acid, (S)-(1 -cyclohexylethyl) amide 10 g (0.0265 mol) and N-bromosuccinimide 9.4 g (0.0528 mol) were suspended in rapidly stirring carbontetrachloride (350 mL). The mixture was warmed to 80° C. in a hot water bath after which time dibenzoyl peroxide (1.28 g, 0.0053 mol) was added in one portion. The mixture was heated at reflux for 30 minutes then cooled rapidly in an ice bath. The resulting suspension was filtered and the filtrate concentrated under reduced pressure. The resulting residue was taken into ethyl acetate and washed with saturated sodium bicarbonate solution, water, brine, and dried over sodium sulfate. Removal of the solvent under reduced pressure provided the crude material which was used in the next step without further purification.

1b) 4-[4-((S)-1-Cyclohexyl-ethylcarbamoyl)-2-thiophen-2-yl-quinolin-3-ylmethyl]-piperazine-1-carboxylic acid tert-butyl ester

To a solution of 3-bromomethyl-2-thiophen-2-yl-quinoline-4-carboxylic acid (S)-(1-cyclohexylethyl)amide (1.4 g, 3.10 mmol) in acetonitrile (100 mL), tert-butyl 1-piperazine-carboxylate (577.4 mg, 3.1 mmol) and K₂CO₃ (861 mg, 6.2 mmol) were added. The reaction mixture was stirred at reflux temperature for 3 hours. The solvent was removed under reduce pressure and the residue was partitioned between ethyl acetate and dilute NaOH(aq.). The combined organic phase was washed with water, brine, dried over sodium sulfate, filtered and concentrated. Column chromatography (30% ethyl acetate:hexanes) of the residue provided 780 mg of the title compound: LC-MS m/z 563.0 (M⁺).

1c) 3-Piperazin-1-ylmethyl-2-thiophen-2-yl-quinoline-4-carboxylic acid ((S)-1-cyclohexyl-ethyl)-amide, HCl salt

HCl in dioxane (4.0M, 16 mL) was added to 4-[4-((S)-1-Cyclohexyl-ethylcarbamoyl)-2-thiophen-2-yl-quinolin-3-ylmethyl]-piperazine-1-carboxylic acid tert-butyl ester (1.5 mg), and the reaction mixture stirred at room temperature for 3 hours. The solvent was evaporated to give the title compound (1.5 g): LC-MS m/z 463.0 (M⁺).

1d) 3-[4-(2-hydroxy-ethanoyl)-piperazin-1-ylmethyl]-2-thiophen-2-yl-quinoline-4-carboxylic acid ((S)-1-cyclohexyl-ethyl)-amide

To the solution of 3-piperazin-1-ylmethyl-2-thiophen-2-yl-quinoline-4-carboxylic acid ((S)-1-cyclohexyl-ethyl)-amide (150 mg, 0.3 mmol) in CH₂Cl₂ (15 mL), 1-(3-dimethylaminopropyl)-3-ethylcarbodiimide (64 mg, 0.33 mmol), glycolic acid (23 mg, 0.3 mmol), 1-hydroxybenzotriazole (40 mg, 0.3 mmol) and triethylamine (0.15 mL, 1.05 mmol) were added. The reaction mixture was stirred at room temperature for 16 hours. Then the reaction mixture was partitioned between CH₂Cl₂ and water. The combined organic phase was washed with brine, dried (MgSO₄), filtered and concentrated. The crude product was purified by Gilson-HPLC to provide 66.8 mg of the title compound. LC-MS m/z 521.0 (M⁺).

### Example 2

### Preparation of 3-[4-((S)-2-hydroxy-propanoyl)-piperazin-1-ylmethyl]-2-thiophen-2-yl-quinoline-4-carboxylic acid ((S)-1-cyclohexyl-ethyl)-amide

Following the general procedure described in Example 1d, 3-Piperazin-1-ylmethyl-2-thiophen-2-yl-quinoline-4-carboxylic acid ((S)-1-cyclohexyl-ethyl)-amide (1.5 g, 3.0 mmol) was coupled with (S)-2-hydroxy-propionic acid (270 mg, 3.0 mmol) to provide 600 mg of the title compound. LC-MS m/z 535.2 (M⁺).

### Example 3

### Preparation of 3-[4-(2-Hydroxy-2-methyl-propanoyl)-piperazin-1-ylmethyl]-2-thiophen-2-yl-quinoline-4-carboxylic acid ((S)-1-cyclohexyl-ethyl)-amide

Following the general procedure described in Example 1d, 3-piperazin-1-ylmethyl-2-thiophen-2-yl-quinoline-4-carboxylic acid ((S)-1-cyclohexyl-ethyl)-amide (200 mg, 0.4 mmol) was coupled with 2-hydroxy-2-methyl-propionic acid (41.6 mg, 0.4 mmol) to provide 140 mg of the title compound. LC-MS m/z 549.2 (M⁺).

### Example 4

### Preparation of 3-[4-((S)-2-hydroxy-3-methyl-butanoyl)-Piperazin-1-ylmethyl]-2-thiophen-2-yl-quinoline-4-carboxylicacid ((S)-1-cyclohexyl-ethyl)-amide

Following the general procedure described in Example 1d, 3-piperazin-1-ylmethyl-2-thiophen-2-yl-quinoline-4-carboxylic acid ((S)-1-cyclohexyl-ethyl)-amide (150 mg, 0.3 mmol) was coupled with (S)-2-hydroxy-3-methyl-butyric acid (35 mg, 0.3 mmol) to provide 80 mg of the title compound. LC-MS m/z 563.4 (M⁺).

### Example 5

### Preparation of 3-[4-((S)-2-cyclohexyl-2-hydroxy-ethanoyl)-piperazin-1-ylmethyll-2-thiophen-2-yl-quinoline-4-carboxylic acid ((S)-1-cyclohexyl-ethyl)-amide

Following the general procedure described in Example 1 d, 3-piperazin-1-ylmethyl-2-thiophen-2-yl-quinoline-4-carboxylic acid ((S)-1-cyclohexyl-ethyl)-amide (150 mg, 0.3 mmol) was coupled with (S)-2-cyclohexyl-2-hydroxy-acetic acid (47.5 mg, 0.3 mmol) to provide 89.4 mg of the title compound. LC-MS m/z 603.2 (M⁺).

### Example 6

### Preparation of 3-{4-[1-((R)-2-hydroxymethyl-pyrrolidin-1-yl)-methanoyl]-piperazin-1-ylmethyl}-2-thiophen-2-yl-quinoline-4-carboxylic acid ((S)-1-cyclohexyl-ethyl)-amide

6a) 4-[4-((S)-1-cyclohexyl-ethylcarbamoyl)-2-thiophen-2-yl-quinolin-3-ylmethyl]-piperazine-1-carbonyl chloride

To a solution of 3-piperazin-1-ylmethyl-2-thiophen-2-yl-quinoline-4-carboxylic acid ((S)-1-cyclohexyl-ethyl)-amide (1.0 g, 2.0 mmol) in CH₂Cl₂ (30 mL), triphosgene (237.4 mg, 0.8 mmol) and triethylamine (0.84 mL, 6.0 mmol) were added at room temperature. The reaction mixture was stirred at room temperature for 1 hour. The solvent was removed under reduced pressure and the residue re-dissolved in ethyl acetate and washed with water. The combined organic phase was washed with brine, dried (MgSO₄), filtered and concentrated to provide 1.0 g of the title compound: LC-MS m/z 525.6 (M⁺).

6b) 3-{4-[1-((R)-2-hydroxymethyl-pyrrolidin-1-yl)-methanoyl]-piperazin-1-ylmethyl]-2-thiophen-2-yl-quinoline-4-carboxylic acid ((S)-1-cyclohexyl-ethyl)-amide

To a solution of 4-[4-((S)-1-cyclohexyl-ethylcarbamoyl)-2-thiophen-2-yl-quinolin-3-ylmethyl]-piperazine-1-carbonyl chloride (230 mg, 0.44 mmol) in CH₂Cl₂ (10 mL), (R)-1-pyrrolidin-2-yl-methanol (44 mg, 0.44 mmol) and triethylamine (0.12 mL, 0.88 mmol) were added. The reaction mixture was stirred at room temperature for 16 hours, then partitioned between CH₂Cl₂ and water. The combined organic phase was washed with brine, dried (MgSO₄), filtered and concentrated. The crude product was purified by Gilson-HPLC to provide 148.3 mg of the title compound. LC-MS m/z 525.6 (M⁺).

### Example 7

### Preparation of 3-{4-[1-((S)-2-hydroxymethyl-pyrrolidin-1-yl)-methanoyl]-piperazin-1-ylmethyl}-2-thiophen-2-yl-quinoline-4-carboxylic acid ((S)-1-cyclohexyl-ethyl)-amide

Following the general procedure described in Example 6b, 4-[4-((S)-1-cyclohexyl-ethylcarbamoyl)-2-thiophen-2-yl-quinolin-3-ylmethyl]-piperazine-1-carbonyl chloride (150 mg, 0.29 mmol) wasa reacted with (S)-1-pyrrolidin-2-yl-methanol (28.9 mg, 0.29 mmol) to provide 125 mg of the title compound. LC-MS m/z 590.2 (M⁺).

### Example 8

### Preparation of 3-{4-[1-(4-hydroxy-piperidin-1-yl)-methanoyl]-piperazin-1-ylmethyl}-2-thiophen-2-yl-quinoline-4-carboxylic acid ((S)-1-cyclohexyl-ethyl)-amide

Following the general procedure described in Example 6b, 4-[4-((S)-1-cyclohexyl-ethylcarbamoyl)-2-thiophen-2-yl-quinolin-3-ylmethyl]-piperazine-1-carbonyl chloride (151 mg, 0.29 mmol) was reacted with piperidin-4-ol (26 mg, 0.29 mmol) to provide 40 mg of the title compound. LC-MS m/z 590.2 (M⁺).

### Example 9

### Preparation of 2-thiophen-2-yl-3-[4-(3,3.3-trifluoro-2-hydroxy-2-methyl-propanoyl)-piperazin-1-ylmethyl]-quinoline-4-carboxylic acid ((S)-1-cyclohexyl-ethyl)-amide

Following the general procedure described in Example 1d, 3-piperazin-1-ylmethyl-2-thiophen-2-yl-quinoline-4-carboxylic acid ((S)-1-cyclohexyl-ethyl)-amide (150 mg, 0.3 mmol) was coupled with 3,3,3-trifluoro-2-hydroxy-2-methyl-propionic acid (47.5 mg, 0.3 mmol) to provide 54.1 mg of the title compound. LC-MS m/z 603.2 (M⁺).

### Example 10

### Preparation of 2-thiophen-2-yl-3-[4-(3.3.3-trifluoro-2-hydroxy-propanoyl)-piperazin-1-ylmethyl]-quinoline-4-carboxylic acid ((S)-1-cyclohexyl-ethyl)-amide

Following the general procedure described in Example 1d, 3-piperazin-1-ylmethyl-2-thiophen-2-yl-quinoline-4-carboxylic acid ((S)-1-cyclohexyl-ethyl)-amide (150 mg, 0.3 mmol) was coupled with 3,3,3-trifluoro-2-hydroxy-propionic acid (43 mg, 0.3 mmol) to provide 45.2 mg of the title compound. LC-MS m/z 589.2 (M⁺).

### Example 11

### Preparation of 2-thiophen-2-yl-3-[4-(4.4.4-trifluoro-3-hydroxy-3-methyl-butanoyl)-piperazin-1-ylmethyl]-quinoline-4-carboxylic acid ((S)-1-cyclohexyl-ethyl)-amide

Following the general procedure described in Example 1d, 3-piperazin-1-ylmethyl-2-thiophen-2-yl-quinoline-4-carboxylic acid ((S)-1-cyclohexyl-ethyl)-amide (150 mg, 0.3 mmol) was coupled with 4,4,4-trifluoro-3-hydroxy-3-methyl-butyric acid (51.6 mg, 0.3 mmol) to provide 116 mg of title the compound. LC-MS m/z 617.2 (M⁺).

### Example 12

### Preparation of 2-thiophen-2-yl-3-[4-(4,4,4-trifluoro-3-hydroxy-butanoyl)-piperazin-1-ylmethyl]-quinoline-4-carboxylic acid ((S)-1-cyclohexyl-ethyl)-amide

Following the general procedure described in Example 1 d, 3-piperazin-1-ylmethyl-2-thiophen-2-yl-quinoline-4-carboxylic acid ((S)-1-cyclohexyl-ethyl)-amide (150 mg, 0.3 mmol) was coupled with 4,4,4-trifluoro-3-hydroxy-butyric acid (47 mg, 0.3 mmol) to provide 116.8 mg of the title compound. LC-MS m/z 603.4 (M⁺).

### Example 13

### Preparation of 3-[4-((S)-2-hydroxy-propanoyl)-2-oxo-piperazin-1-ylmethyl]-2-thiophen-2-yl-quinoline-4-carboxylic acid ((S)-1-cyclohexyl-ethyl)-amide

Following the general procedure described in Example 1d, 3-(2-oxo-piperazin-1-ylmethyl)-2-thiophen-2-yl-quinoline-4-carboxylic acid ((S)-1-cyclohexyl-ethyl)-amide (150 mg, 0.3 mmol) was coupled with (S)-2-hydroxy-propionic acid (47 mg, 0.3 mmol) to provide 27.9 mg of the title compound. LC-MS m/z 549.4 (M⁺).

What is claimed is:

1. A compound according to formula (I) 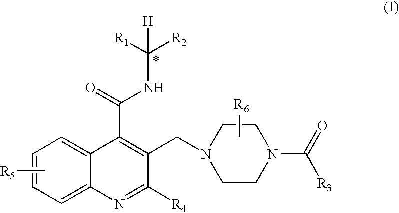
wherein:
R₁ is H or (C₁₋₆)alkyl;
R₂ is aryl, (C₃₋₇)cycloalkyl, or heterocycle; 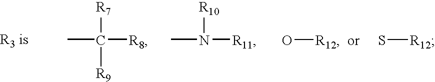
R₄ is phenyl or heterocycle;
R₅ is H or up to three substitutents independently selected from the list consisting of (C₁₋₆)alkyl, (C₂₋₆)alkenyl, aryl, alkoxy, or a hydroxylated deriviative thereof, hydroxy, halogen, nitro, cyano, carboxy, alkylcarboxy, alkylcarboxyalkyl, haloalkyl, and amino or mono- or dialkylamino; or R₅ represents a bridging moiety which is arranged to bridge two adjacent ring atoms wherein the bridging moiety comprises alkyl or dioxyalkylene;
R₆ is absent or oxo;
R₇ is —OH or (C₁₋₆)alkylOH;
R₈ and R₉ are each independently H, (C₁₋₆)alkyl, (C₃₋₇)cycloalkyl, aryl, or heterocycle;
R₁₀ and R₁₁ together with the N atom form a heterocycle ring which is substituted by —OH, or —(C₁₋₆)alkylOH;
R₁₂ is H, (C₁₋₆)alkyl, aryl, or heterocycle; or a pharmaceutically acceptable salt thereof.

2. A compound according to claim 1 wherein R₁ is methyl.

3. A compound according to claim 1 wherein R₂ is (C₃₋₇)cycloalkyl.

4. A compound according to claim 1 wherein R₄ is 2- or 3-thiophene.

5. A compound according to claim 1 wherein R₇ is —OH or (C₁₋₆)alkylOH unsubstituted or substituted by one to three halo groups.

6. A compound according to claim 1 wherein R₉ is H and R₈ is H, unsubstituted C(₃₋₇)cycloalkyl, or (C₁₋₆)alkyl unsubstituted or substituted by one to five substituents selected from the group consisting of halo and —OH.

7. A compound according to claim 1 wherein R₁₀ and R₁₁ together with the N atom form pyrrolidine substituted by —OH or —(C₁₋₆)alkylOH or piperidine substituted by —OH or —(C₁₋₆)alkylOH.

8. A compound according to claim 1 which is:
3-[4-(2-Hydroxy-ethanoyl)-piperazin-1-ylmethyl]-2-thiophen-2-yl-quinoline-4-carboxylic acid ((S)-1-cyclohexyl-ethyl)-amide;
3-[4-((S)-2-Hydroxy-propanoyl)-piperazin-1-ylmethyl]-2-thiophen-2-yl-quinoline-4-carboxylic acid ((S)-1-cyclohexyl-ethyl)-amide;
3-[4-(2-Hydroxy-2-methyl-propanoyl)-piperazin-1-ylmethyl]-2-thiophen-2-yl-quinoline-4-carboxylic acid ((S)-1-cyclohexyl-ethyl)-amide;
3-[4-((S)-2-Hydroxy-3-methyl-butanoyl)-piperazin-1-ylmethyl]-2-thiophen-2-yl-quinoline-4-carboxylicacid ((S)-1-cyclohexyl-ethyl)-amide;
3-[4-((S)-2-Cyclohexyl-2-hydroxy-ethanoyl)-piperazin-1-ylmethyl]-2-thiophen-2-yl-quinoline-4-carboxylic acid ((S)-1-cyclohexyl-ethyl)-amide;
3-{4-[1-((R)-2-Hydroxymethyl-pyrrolidin-1-yl)-methanoyl]-piperazin-1-ylmethyl}-2-thiophen-2-yl-quinoline-4-carboxylic acid ((S)-1-cyclohexyl-ethyl)-amide;
3-{4-[1-((S)-2-Hydroxymethyl-pyrrolidin-1-yl)-methanoyl]-piperazin-1-ylmethyl}-2-thiophen-2-yl-quinoline-4-carboxylic acid ((S)-1-cyclohexyl-ethyl,-amide;
3-{4-[1-(4-Hydroxy-piperidin-1-yl)-methanoyl]-piperazin-1-ylmethyl}-2-thiophen-2-yl-quinoline-4-carboxylic acid ((S)-1-cyclohexyl-ethyl)-amide;
2-Thiophen-2-yl-3-[4-(3,3,3-trifluoro-2-hydroxy-2-methyl-propanoyl)-piperazin-1-ylmethyl]-quinoline-4-carboxylic acid ((S)-1-cyclohexyl-ethyl)-amide;
2-Thiophen-2-yl-3-[4-(3,3,3-trifluoro-2-hydroxy-propanoyl)-piperazin-1-ylmethyl]-quinoline-4-carboxylic acid ((S)-1-cyclohexyl-ethyl)-amide;
2-Thiophen-2-yl-3-[4-(4,4,4-trifluoro-3-hydroxy-3-methyl-butanoyl)-piperazin-1-ylmethyl]-quinoline-4-carboxylic acid ((S)-1-cyclohexyl-ethyl)-amide;
2-Thiophen-2-yl-3-[4-(4,4,4-trifluoro-3-hydroxy-butanoyl)-piperazin-1-ylmethyl]-quinoline-4-carboxylic acid ((S)-1-cyclohexyl-ethyl)-amide; or
3-[4-((S)-2-Hydroxy-propanoyl)-2-oxo-piperazin-1-ylmethyl]-2-thlophen-2-yl-quinoline-4-carboxylic acid ((S)-1-cyclohexyl-ethyl)-amide; or a pharmaceutically acceptable salt thereof.

9. A pharmaceutical composition which comprises a compound according to claim 1 and a pharmaceutically acceptable carrier.

10. A method for the treatment of the Primary and Secondary conditions in mammals, particularly humans, which comprises administering to a subject in need of such treatment an effective amount of a compound of formula (I) or a pharmaceutically acceptable salt thereof.

11. A method for the treatment of respiratory diseases in mammals, which comprises administering, to a subject in need of such treatment, an effective amount of a compound according to formula (I) or a pharmaceutically acceptable salt thereof.
